(19) United States
(12) Patent Application Publication
(10) Pub. No.: US 2022/0293258 A1
(43) Pub. Date: Sep. 15, 2022

(54) EVALUATION DEVICE AND METHOD FOR BLOOD HEALTH, AND ASSESSMENT METHOD FOR DAMAGE CONTRIBUTION DEGREE OF PHYSIOLOGICAL INDEX

(71) Applicant: BOE TECHNOLOGY GROUP CO., LTD., Beijing (CN)

(72) Inventor: Xu ZHANG, Beijing (CN)

(21) Appl. No.: 17/633,990

(22) PCT Filed: Mar. 9, 2021
(86) PCT No.: PCT/CN2021/079640

§ 371 Date: Feb. 9, 2022

(30) Foreign Application Priority Data
Apr. 8, 2020 (CN) 2020 10267253.0

(51) Int. Cl. G16H 40/63

(52) U.S. Cl. CPC G16H 40/63

(57) ABSTRACT

An evaluation device (100) for a health condition of a blood circulatory system, an assessment method for a damage contribution degree of a physiological index, and an assessment method for a health condition of a blood circulatory system are provided. The evaluation device (100) for a health condition of a blood circulatory system includes: a module (110) to assess a damage contribution degree of a physiological index and a module (120) to assess a blood health. The device is capable of performing direct analysis and assessment on contribution degree of each of the physiological indexes to the health damage of a human body and health condition of the blood circulatory system.

### TECHNICAL FIELD

The present disclosure relates to a medical field, and specifically relates to an evaluation device for a health condition of a blood circulatory system, an assessment method for a damage contribution degree of a physiological index, and an assessment method for a health condition of a blood circulatory system.

### BACKGROUND

In the existing art, a plurality of physiological indexes is defined to characterize a physiological state of a human body. When a health state of a human body needs to be assessed, a particular device is used for detecting the respective physiological indexes and acquiring the physiological data corresponding to the physiological indexes. Meanwhile, each of the physiological indexes has a corresponding recommended value range for judging whether the physiological data corresponding to the physiological index is normal. That is, the human body is considered as healthy only when the physiological data falls into the recommended value range.

At present, after a particular device is used to detect and acquire the physiological data corresponding to the respective physiological indexes, there is a need for a method and a device which can accurately assess an influence of the physiological data on human health.

### SUMMARY

In order to solve at least one of the above problems in the existing art, the present disclosure provides an evaluation device for a health condition of a blood circulatory system, an assessment method for a damage contribution degree of a physiological index, and an assessment method for a health condition of a blood circulatory system.

To achieve the above objects, as a first aspect of the present disclosure, there is provided an evaluation device for a health condition of a blood circulatory system, including:

a module to assess a damage contribution degree of a physiological index, configured to calculate, according to a measured value of a physiological index to be analyzed of a subject, a damage contribution degree score of the physiological index to be analyzed, the damage contribution degree score characterizing a contribution degree of the physiological index to be analyzed to a health damage of the subject; and

a module to assess a blood health, configured to determine, according to the damage contribution degree score of each of the physiological indexes related to the health condition of a blood circulatory system of the subject that is calculated and obtained by the module to assess the damage contribution degree of the physiological index, the health condition of the blood circulatory system of the subject.

Optionally, the module to assess a damage contribution degree of a physiological index includes:

a basic parameter assignment unit configured to determine a value of at least one basic parameter of the physiological index to be analyzed, different basic parameters characterizing different clinical conclusions of the physiological index to be analyzed; and

a damage contribution degree score calculation unit configured to calculate, according to the value of the basic parameter and the measured value of the physiological index to be analyzed of the subject, a damage contribution degree score of the physiological index to be analyzed of the subject.

Optionally, the basic parameter includes a top value and a bottom value; values of the physiological index to be analyzed are distributed in a plurality of intervals, including a healthy interval and a plurality of non-healthy intervals,

the top value is an upper limit of the healthy interval; and

the bottom value is a lower limit of the healthy interval.

Optionally, the basic parameter further includes an increased region and a decreased region;

the increased region represents a difference between the upper limit value and the lower limit value in the non-healthy interval of which the lower limit value is above the upper limit of the healthy interval; and

the decreased region represents a difference between the upper limit value and the lower limit value in the non-healthy interval of which the upper limit value is below the lower limit of the healthy interval.

Optionally, the damage contribution degree score increases non-linearly with increase of a deviation magnitude of the measured value from a normal value range of the physiological index to be analyzed.

Optionally, the damage contribution degree score is calculated by:

$$F = \{ \begin{matrix}1 - power ( 2 , \frac{Value - {R}_{C}}{α} ) , & Value > {R}_{C} \\ 1 - power ( 2 , \frac{{R}_{F} - Value}{β} ) , & Value < {R}_{F} \\ 0 , & {R}_{F} \leq Value \leq {R}_{C}\end{matrix}$$

where F is the damage contribution degree score, Value is the measured value, R_{C} is the top value, R_{F} is the bottom value, α is the increased region, and β is the decreased region.

Optionally, the module to assess a blood health includes:

a blood health state index calculation unit configured to calculate each blood health state index according to the calculated damage contribution degree score of each of the physiological indexes, each blood health state index characterizing health condition of each of the blood circulatory subsystems; and

a blood health index calculation unit configured to calculate a blood health index according to each calculated blood health state index, the blood health index characterizing health condition of the blood circulatory system.

Optionally, the blood health state index calculation unit includes:

a weight assignment subunit configured to determine a weight of each of the physiological indexes occupied in each blood health state index, respectively; and

a blood health state index calculation subunit configured to respectively calculate each of the blood health state index, according to the damage contribution degree score of each of the physiological indexes and the weight of each of the physiological indexes occupied in each blood health state index.

Optionally, the blood health state index includes a pulmonary blood circulation index, a cardiovascular state index, a blood component state index, and a renal blood metabolism index, and the blood health state index is calculated by:

Pⱼ=ΣFᵢ×ωᵢ×100

where Pⱼ is the blood health state index, Fᵢ is the damage contribution degree score of the physiological index, ωᵢ is the weight of the physiological index Fᵢ occupied in the blood health state index Pⱼ, i and j are positive integers, different values of i correspond to different physiological indexes, and different values of j correspond to different blood health state indexes.

Optionally, the blood health state index includes a pulmonary blood circulation index, a cardiovascular state index, a blood component state index, and a renal blood metabolism index, and the blood health index is calculated by:

BHI=[2×min(P₁, P₂, P₃, P₄)+P₁+P₂+P₃+P₄]/6

where BHI is a blood health index, P₁ is a pulmonary blood circulation index, P₂ is a cardiovascular state index, P₃ is a blood component state index, and P₄ is a renal blood metabolism index.

As a second aspect of the present disclosure, there is provided an assessment method for a damage contribution degree of a physiological index, including:

determining a value of at least one basic parameter of the physiological index to be analyzed, the different basic parameters characterizing different clinical conclusions of the physiological index to be analyzed; and

calculating, according to the value of the basic parameter and the measured value of the physiological index to be analyzed of the subject, a damage contribution degree score of the physiological index to be analyzed of the subject, the damage contribution degree score characterizing a contribution degree of the physiological index to be analyzed to the health damage of the subject.

Optionally, the basic parameter includes a top value and a bottom value;

values of the physiological index to be analyzed are distributed in a plurality of intervals, including a healthy interval and a plurality of non-healthy intervals,

the top value is an upper limit of the healthy interval; and

the bottom value is a lower limit of the healthy interval.

Optionally, the basic parameter further includes an increased region and a decreased region;

the increased region represents a difference between the upper limit value and the lower limit value in a non-healthy interval of which the lower limit value is above the upper limit of the healthy interval; and

the decreased region represents a difference between the upper limit value and the lower limit value in a non-healthy interval of which the upper limit value is below the lower limit of the healthy interval.

Optionally, the damage contribution degree score increases non-linearly with increase of a deviation magnitude of the measured value from a normal value range of the physiological index to be analyzed.

Optionally, the damage contribution degree score is calculated by:

$$F = \{ \begin{matrix}1 - power ( 2 , \frac{Value - {R}_{C}}{α} ) , & Value > {R}_{C} \\ 1 - power ( 2 , \frac{{R}_{F} - Value}{β} ) , & Value < {R}_{F} \\ 0 , & {R}_{F} \leq Value \leq {R}_{C}\end{matrix}$$

where F is the damage contribution degree score, Value is the measured value, R_{C} is the top value, R_{F} is the bottom value, α is the increased region, and β is the decreased region.

As a third aspect of the present disclosure, there is provided an assessment method for a health condition of a blood circulatory system, including:

calculating, according to the method for assessing the damage contribution degree of a physiological index provided in the present disclosure as described above, a damage contribution degree score of each of the physiological indexes related to the health condition of the blood circulatory system of the subject; and

determining, according to the damage contribution degree score of each of the physiological indexes of the subject, the health condition of the blood circulatory system of the subject.

Optionally, the determining, according to the damage contribution degree score of each of the physiological indexes of the subject, the health condition of the blood circulatory system of the subject includes:

calculating each blood health state index according to the calculated damage contribution degree score of each of the physiological indexes, each blood health state index characterizing health condition of each of the blood circulatory subsystems; and

calculating a blood health index according to each calculated blood health state index, the blood health index characterizing health condition of the blood circulatory system.

Optionally, the calculating each blood health state index according to the calculated damage contribution degree score of each of the physiological indexes includes:

determining a weight of each of the physiological indexes occupied in each of the blood health state index, respectively; and

respectively calculating each blood health state index according to the damage contribution degree score of each of the physiological indexes and the weight of each of the physiological indexes occupied in each blood health state index.

Optionally, the blood health state index includes a pulmonary blood circulation index, a cardiovascular state index, a blood component state index, and a renal blood metabolism index, and the blood health state index is calculated by:

Pⱼ=ΣFᵢ×ωᵢ+100

where Pⱼ is the blood health state index, Fᵢ is the damage contribution degree score of the physiological index, ωᵢ is the weight of the physiological index Fᵢ occupied in the blood health state index P, i and j are positive integers, different values of i correspond to different physiological indexes, and different values of j correspond to different blood health state indexes.

Optionally, the blood health state index includes a pulmonary blood circulation index, a cardiovascular state index, a blood component state index, and a renal blood metabolism index, and the blood health index is calculated by:

BHI=[2×min(P₁, P₂, P₃, P₄)+P₁+P₂+P₃+P₄]/6

where BHI is the blood health index, P₁ is the pulmonary blood circulation index, P₂ is the cardiovascular state index, P₃ is the blood component state index, and P₄ is the renal blood metabolism index.

### BRIEF DESCRIPTION OF DRAWINGS

Accompanying drawings are provided for further understanding of this disclosure and constitute a part of the specification. Hereinafter, these drawings are intended to explain the disclosure together with the following specific embodiments, but should not be considered as a limitation of the disclosure. In the drawings:

FIG. 1 is a block diagram of an implementation of an evaluation device provided by the present disclosure;

FIG. 2 is a block diagram of another implementation of an evaluation device provided by the present disclosure;

FIG. 3 is a block diagram of another implementation of an evaluation device provided by the present disclosure;

FIG. 4 is a block diagram of another further implementation of an evaluation device provided by the present disclosure;

FIG. 5 is a block diagram of another further implementation of an evaluation device provided by the present disclosure;

FIG. 6 is a flowchart of an implementation of an assessment method for a contribution degree of a physiological index provided by the present disclosure;

FIG. 7 is a flowchart of an implementation of an assessment method provided by the present disclosure;

FIG. 8 is a flowchart of an implementation of another assessment method provided by the present disclosure;

FIG. 9 is a flowchart of an implementation of another assessment method provided by the present disclosure;

FIG. 10 is a flowchart of an implementation of another assessment method provided by the present disclosure;

FIG. 11 is a schematic diagram of an implementation of the damage contribution degree score in the present disclosure; and

FIG. 12 is a schematic diagram of an implementation of the blood health radar map in the present disclosure.

### DETAIL DESCRIPTION OF EMBODIMENTS

Hereinafter, specific embodiments of the present disclosure will be described with respect to the accompanying drawings. It should be understood that the specific embodiments as set forth herein are merely for the purpose of illustration and explanation of the invention and should not be constructed as a limitation thereto.

The inventor of the present disclosure has studied and found that after the physiological data corresponding to the respective physiological indexes are detected and acquired by using a particular device, the subject can only determine whether each piece of physiological data is normal by comparing the data with a recommended value, but cannot determine the impact level of each piece of physiological data on human health. In addition, without professional medical knowledge, the subject cannot visually assess the overall health condition even based on various physiological data obtained by measurement.

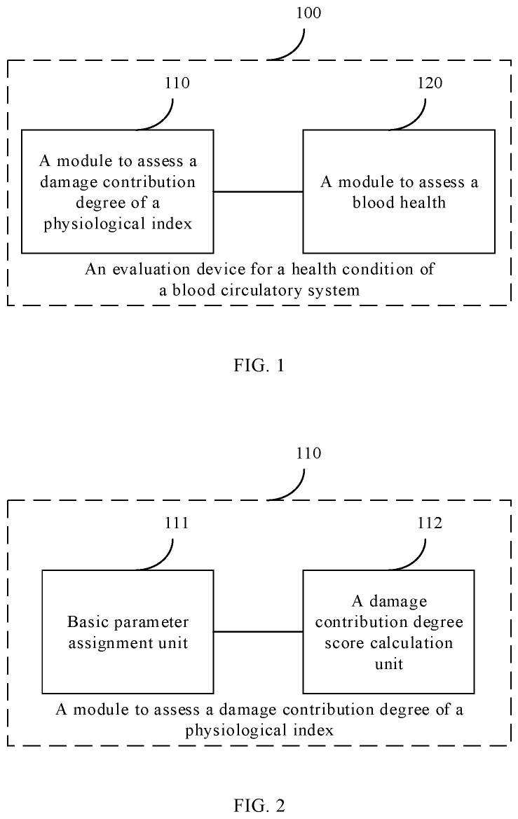

In view of this, as a first aspect of the present disclosure, as shown in FIG. 1, there is provided an evaluation device 100 for a health condition of a blood circulatory system, including:

a module 110 to assess a damage contribution degree of a physiological index, configured to calculate, according to a measured value of a physiological index to be analyzed of a subject, a damage contribution degree score of the physiological index to be analyzed, the damage contribution degree score characterizing the contribution degree of the physiological index to be analyzed to the health damage of the subject; and

a module 120 to assess a blood health, configured to determine, according to a damage contribution degree score of each of the physiological indexes related to the health condition of a blood circulatory system of the subject that is calculated and obtained by the module to assess a damage contribution degree of a physiological index, the health condition of the blood circulatory system of the subject.

Each of the physiological indexes has a corresponding normal value range. When the measured value falls within the normal value range, it indicates that the physiological index is normal; and when the measured value deviates from the normal value range, it indicates that the physiological index is abnormal. When the health of the subject is damaged, the health damage degree of the subject is evaluated by measuring various physiological indexes. For each of the physiological indexes, different physiological indexes may not have the same contribution degree to the health damage of the subject, and different measurement results of the same physiological index may have different contribution degree to the health damage of the subject. A damage contribution degree score of each of the physiological indexes is defined in the evaluation device 100 provided by the present disclosure, which is configured to characterize the contribution degree of the physiological index to the health damage of the subject when the value of the physiological index changes. After the measured value of a physiological index to be analyzed of the subject is obtained, a damage contribution degree score of the physiological index to be analyzed is calculated in conjugation with the clinical significance, and thus the contribution degree of the physiological index to be analyzed to the health damage of the subject can be visually assessed through the damage contribution degree score.

In the present disclosure, the specific mode of the damage contribution degree score is not particularly limited. For example, a linear assessment mode may be adopted, which means the damage contribution degree score increases or decreases linearly with a deviation magnitude of the measured value of the physiological index to be analyzed from the normal value range; or a non-linear assessment mode may be adopted, which means the damage contribution degree score increases or decreases non-linearly with the deviation magnitude of the measured value of the physiological index to be analyzed from the normal value range.

There are various physiological indexes related to the health of the blood circulatory system, of which the damage contribution degree scores can be calculated by the module to assess a damage contribution degree of a physiological index 110. Further, the module to assess a blood health 120 determines, according to the damage contribution degree score of each of the physiological indexes related to the health of the blood circulatory system of the subject that is calculated and obtained by the module to assess a damage contribution degree of a physiological index 110, the health condition of the blood circulatory system of the subject.

The evaluation device for a health condition of a blood circulatory system provided by the present disclosure calculates the damage contribution degree score by combining the measured value of the physiological index of the subject and the basic parameter value determined according to the clinical significance so that when the health of the subject is damaged, the contribution degree of a change in the measured value of each of the physiological indexes to the health damage of the subject can be visually analyzed and assessed, such that the subject can more clearly know the clinical significance of each of the physiological indexes. The evaluation device further calculates and obtains a blood health index according to the damage contribution degree score of each of the physiological indexes related to the health condition of the blood circulatory system such that that a visual and comprehensive assessment of the health condition of the blood circulatory system is achieved, and the health condition of the subject per se can conveniently and visually understood.

The basic parameters for calculating the damage contribution degree score are defined in the module to assess a damage contribution degree of a physiological index 110 of the present disclosure. It should be noted that each of the physiological indexes has a corresponding normal value range, and the characteristic of different physiological indexes have different clinical significances. Therefore, when the module to assess a damage contribution degree of a physiological index 110 calculates the damage contribution degree score of the physiological index to be analyzed, the value of the basic parameter needs to be determined in combination with the clinical significance of the physiological index to be analyzed. Accordingly, as shown in FIG. 2, the module to assess a damage contribution degree of a physiological index 110 includes:

a basic parameter assignment unit 111 configured to determine a value of at least one basic parameter of the physiological index to be analyzed, the different basic parameters characterizing the different clinical conclusions of the physiological index to be analyzed; and

a damage contribution degree score calculation unit 112 configured to calculate, according to the value of the basic parameter and the measured value of the physiological index to be analyzed of the subject, a damage contribution degree score of the physiological index to be analyzed of the subject.

As described above, each of the physiological indexes has a corresponding normal value range, and when the measured value falls within the normal value range, it indicates that the physiological index of the subject is normal, so this physiological index does not contribute to health damage of the subject. When the measured value does not fall within the normal value range, it indicates that the physiological index of the subject is abnormal, so this physiological index contributes to the health damage of the subject. Therefore, it is clinically significant to divide the range during which the value of the physiological index may vary into a healthy interval and a non-healthy interval, and to assess the contribution degree of the physiological index to the health damage of the subject on this basis.

In the present disclosure, the term “non-healthy” in the non-healthy interval is not particularly limited. A “non-healthy” state may be a “sub-healthy” state or may be a “diseased state”.

Accordingly, as an optional implementation, the basic parameter includes a top value and a bottom value.

Values of the physiological index to be analyzed are distributed in a plurality of intervals, including a healthy interval and a plurality of non-healthy intervals.

The top value is an upper limit of the healthy interval; and

the bottom value is a lower limit of the healthy interval.

The inventor of the present disclosure has studied and found that different intervals of the physiological index reflect different clinical conclusions. Taking the systolic blood pressure as an example, the interval between 90 and 120 mmHg represents a normal blood pressure, the interval between 140 and 160 mmHg represents mild hypertension, the interval between 160 and 180 mmHg represents moderate hypertension, the interval greater than 180 mmHg represents severe hypertension, and the interval of less than 90 mmHg represents hypotension. It may be further refined, for example, a too low interval represents potential shock. Therefore, the present disclosure may, on the basis of dividing the range during which the value of the physiological index may vary into a healthy interval and a non-healthy interval, further use the relevant characteristic of the intervals representing different clinical conclusions as the basic parameters for calculating the damage contribution degree score in the module to assess a damage contribution degree of a physiological index 110.

Accordingly, as an optional implementation, the basic parameter further includes an increased region and a decreased region;

the increased region represents a difference between the upper limit value and the lower limit value in a non-healthy interval of which the lower limit value is above the upper limit of the healthy interval; and

the decreased region represents a difference between the upper limit value and the lower limit value of a non-healthy interval of which the upper limit value is below the lower limit of the healthy interval.

It should be noted that the lengths of the intervals above the healthy interval corresponding to different clinical conclusions may be not the same as the lengths of the intervals below the healthy interval corresponding to different clinical conclusions. Therefore, in the present disclosure, the increased region and the decreased region are assigned with values respectively, and the values of the increased region and the decreased region may be different or may be the same according to different clinical conclusions of different physiological indexes. Therefore, different clinical conclusions characterized by different physiological indexes in different value intervals can be more accurately reflected, and more accurate damage contribution degree scores can be obtained to precisely assess the damage degree of the physiological indexes to the health.

As an optional implementation, when the physiological index to be analyzed is systolic blood pressure, the healthy interval of the physiological index to be analyzed is [90, 120], the increased region is 20, and the decreased region is 10, in units of mmHg.

Table 1 gives an optional implementation of assignment of basic parameters and the corresponding measured values of 14 physiological indexes of a man.

TABLE 1

| | | Measured | Top | Bottom | Increased | Decreased |
|---|---|---|---|---|---|---|
| Abbreviation | Name | value | value | value | region | region |
| SBP | Systolic blood | 124 | 120 | 90 | 20 | 10 |
| | pressure | | | | | |
| DBP | Diastolic blood | 73 | 80 | 60 | 10 | 5 |
| | pressure | | | | | |
| MAP | Mean arterial | 84 | 105 | 75 | 10 | 5 |
| | pressure* | | | | | |
| PPR | Peripheral pulse | 72 | 100 | 55 | 30 | 5 |
| | rate | | | | | |
| CO | Cardiac output* | 5.7 | 8 | 4 | 1 | 0.5 |
| SV | Stroke volume | 79.8 | 100 | 60 | 10 | 5 |
| PO2 | Oxygen partial | 70 | 105 | 75 | 5 | 5 |
| | pressure | | | | | |
| O2 | Oxygen content | 15.8 | 23 | 15 | 1 | 1 |
| SPO2 | Saturation of blood | 92 | 100 | 98 | 1 | 5 |
| | oxygen | | | | | |
| PCO2 | CO₂ partial pressure | 53 | 48 | 32 | 20 | 6 |
| TCO2 | Total CO₂ | 49 | 32 | 24 | 20 | 3 |
| pH | Power of hydrogen | 7.37 | 7.45 | 7.35 | 0.025 | 0.025 |
| Hb | Haemoglobin | 16 | 17.5 | 13.5 | 1 | 1.5 |
| RBC | Red blood cell | 4.81 | 5.7 | 4.3 | 0.3 | 0.3 |
| | count | | | | | |
| HCT | Hematokrit | 47.2 | 49 | 39 | 2.5 | 1.5 |
| BV | Blood viscosity | 75 | 85 | 65 | 1.5 | 3 |
| G | Gender | Male | x | x | x | x |

Table 2 gives an optional implementation of assignment of basic parameters and the corresponding measured values of 14 physiological indexes of a woman.

TABLE 2

| | | Measured | Top | Bottom | Increased | Decreased |
|---|---|---|---|---|---|---|
| Abbreviation | Name | value | value | value | zone | zone |
| SBP | Systolic blood | 124 | 120 | 90 | 20 | 10 |
| | pressure | | | | | |
| DBP | Diastolic blood | 73 | 80 | 60 | 10 | 5 |
| | pressure | | | | | |
| MAP | Mean arterial | 84 | 105 | 75 | 10 | 5 |
| | pressure* | | | | | |
| PPR | Peripheral pulse | 72 | 100 | 55 | 30 | 5 |
| | rate | | | | | |
| CO | Cardiac output* | 5.7 | 8 | 4 | 1 | 0.5 |
| SV | Stroke volume | 79.8 | 100 | 60 | 10 | 5 |
| PO2 | Oxygen partial | 70 | 105 | 75 | 5 | 5 |
| | pressure | | | | | |
| O2 | Oxygen content | 15.8 | 23 | 15 | 1 | 1 |
| SPO2 | Saturation of blood | 92 | 100 | 98 | 1 | 5 |
| | oxygen | | | | | |
| PCO2 | CO₂ partial pressure | 53 | 48 | 32 | 20 | 6 |
| TCO2 | Total CO₂ | 49 | 32 | 24 | 20 | 3 |
| pH | Power of hydrogen | 7.37 | 7.45 | 7.35 | 0.025 | 0.025 |
| Hb | Haemoglobin | 16 | 16 | 12 | 1 | 1.5 |
| RBC | Red blood cell | 4.81 | 5.1 | 3.8 | 0.3 | 0.3 |
| | count | | | | | |
| HCT | Hematokrit | 47.2 | 45 | 35 | 2.5 | 1.5 |
| BV | Blood viscosity | 75 | 85 | 65 | 1.5 | 3 |
| G | Gender | Female | x | x | x | x |

It will be appreciated that the larger the deviation magnitude of the physiological index from the normal value range, the greater the contribution degree to the health damage of the subject will be. In order to reflect the above clinical significance of the physiological index, as an optional implementation, as shown in FIG. 11, in the module to assess a damage contribution degree of a physiological index 110, when the damage contribution degree score is 0, it means that the physiological index to be analyzed does not contribute to the health damage of the subject; and

when the damage contribution degree score is greater than 0, the damage contribution degree score is positively correlated with the contribution degree of the physiological index to be analyzed to the health damage of the subject.

The inventor of the present disclosure has studied and found that the contribution degree of the physiological index to the health damage of the subject is not linearly increased along with the increase of the deviation magnitude of the physiological index from the normal value range. In the present disclosure, the relationship between the deviation magnitude of the physiological index from the normal value range and the contribution degree to the health damage of the subject is reflected by the non-linear increase of the damage contribution degree score.

Accordingly, as an optional implementation, as shown in FIG. 11, the damage contribution degree score increases non-linearly with increase of the deviation magnitude of the measured value from the normal value range of the physiological index to be analyzed.

As an optional implementation, the damage contribution degree score increases more rapidly as the deviation magnitude of the physiological index from the normal range increases.

As an optional implementation, in the step 120, the damage contribution degree score is calculated by equation (4):

$$F = \{ \begin{matrix}1 - power ( 2 , \frac{Value - {R}_{C}}{α} ) , & Value > {R}_{C} \\ 1 - power ( 2 , \frac{{R}_{F} - Value}{β} ) , & Value < {R}_{F} \\ 0 , & {R}_{F} \leq Value \leq {R}_{C}\end{matrix} \qquad ( 4 )$$

where F is the damage contribution degree score, Value is the measured value, R_{C} is the top value, R_{F} is the bottom value, α is the increased region, and β is the decreased region.

In order to comprehensively assess the health condition of the blood circulatory system, both of the overall health condition of the whole blood circulatory system and the health condition of each blood circulatory subsystem should be assessed. In the present disclosure, the blood health state index is defined to respectively characterize the health condition of each subsystem in the blood circulation, and the blood health index is further calculated from the corresponding blood health state index to assess the overall health condition of the whole blood circulatory system.

Accordingly, as shown in FIG. 3, the module to assess a blood health 120 includes:

a blood health state index calculation unit 121 configured to calculate each blood health state index according to the calculated damage contribution degree score of each of the physiological indexes, each blood health state index characterizing the health condition of each of blood circulatory subsystems; and

a blood health index calculation unit 122 configured to calculate a blood health index according to each of the calculated blood health state indexes, the blood health index characterizing the health condition of the blood circulatory system.

Among various subsystems in the blood circulation, the same physiological index may have different influences on different subsystems; and in the same subsystem, different physiological indexes may also have different influences on the subsystem. In the present disclosure, each of the physiological indexes is assigned with a weight value in each subsystem in combination with the clinical significance, which reflects the weight of the damage contribution degree of each of the physiological indexes to each subsystem in the blood circulation when the subsystem suffers from health damage.

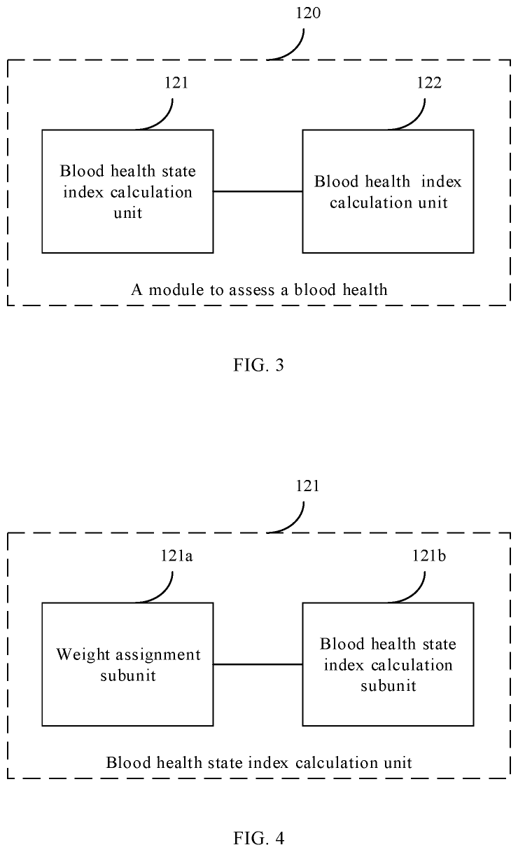

Accordingly, as shown in FIG. 4, the blood health state index calculation unit 121 includes:

a weight assignment subunit 121a configured to determine a weight of each of the physiological indexes occupoied in each blood health state index, respectively; and

a blood health state index calculation subunit 121b configured to respectively calculate each blood health state index according to the damage contribution degree score of each of the physiological indexes and the weight of each of the physiological indexes in each blood health state index.

As an optional implementation, in the blood health state index calculation subunit 121b, the blood health state index includes a pulmonary blood circulation index, a cardiovascular state index, a blood component state index, and a renal blood metabolism index, and the blood health state index is calculated by equation (2):

Pⱼ=ΣFᵢ×ωᵢ+100 (2)

where Pⱼ is the blood health state index, Fᵢ is the damage contribution degree score of the physiological index, ωᵢ is the weight of the physiological index Fᵢ occupied in the blood health state index Pᵢ, i and j are positive integers, the different values of i correspond to the different physiological indexes, and the different values of j correspond to the different blood health state indexes.

Table 3 gives an optional implementation of the damage contribution degree scores of 14 physiological indicators and the weight of each of the physiological indexes occupied in each blood health state index.

TABLE 3

| | | | Weight | | | |
|---|---|---|---|---|---|---|
| | | | Pulmonary | | | Renal |
| | | Damage | blood | | Blood | blood |
| | | contribution | circulation | Cardiovascular | component | metabolism |
| Abbreviation | Name | degree score | index | condition index | state index | index |
| SBP | Systolic blood | −0.15 | — | 7 | — | — |
| | pressure | | | | | |
| DBP | Diastolic blood | 0.00 | — | 10 | — | — |
| | pressure | | | | | |
| MAP | Mean arterial | 0.00 | — | 7 | — | 5 |
| | pressure* | | | | | |
| PPR | Peripheral pulse | 0.00 | — | 7 | — | — |
| | rate | | | | | |
| CO | Cardiac output* | 0.00 | — | 7 | — | — |
| SV | Stroke volume | 0.00 | — | 10 | — | — |
| PO2 | Oxygen partial | −1.00 | 7 | — | — | — |
| | pressure | | | | | |
| O2 | Oxygen content | 0.00 | 7 | — | 5 | — |
| SPO2 | Saturation of | −1.30 | 10 | — | — | — |
| | bloodl oxygen | | | | | |
| PCO2 | CO₂ partial | −0.19 | 7 | — | — | — |
| | pressure | | | | | |
| TCO2 | Total CO₂ | −0.80 | 7 | — | 5 | — |
| pH | Power of | 0.00 | 10 | — | 7 | 10 |
| | hydrogen | | | | | |
| Hb | Haemoglobin | 0.00 | 4 | — | 7 | — |
| RBC | Red blood cell | 0.00 | 4 | — | 7 | — |
| | count | | | | | |
| HCT | Hematokrit | −0.84 | 4 | — | 7 | 7 |
| BV | Blood viscosity | 0.00 | — | 10 | 10 | 7 |
| G | Gender | — | — | — | — | — |

After the blood health state index calculation unit 121 calculates to obtain the pulmonary blood circulation index, the cardiovascular state index, the blood component state index, and the renal blood metabolism index, the blood health index calculation unit 122 calculates a blood health index according to each of the calculated blood health state index.

As an optional implementation, in the blood health index calculation unit 122, the blood health index is calculated by equation (3):

BHI=[2×min(P₁, P₂, P₃, P₄)+P₁+P₂+P₃+P₄]/6

where BHI is a blood health index, P₁ is a pulmonary blood circulation index, P₂ is a cardiovascular state index, P₃ is a blood component state index, and P₄ is a renal blood metabolism index.

In the present disclosure, the health condition of the blood circulatory system is more visually presented by constructing a blood health radar map.

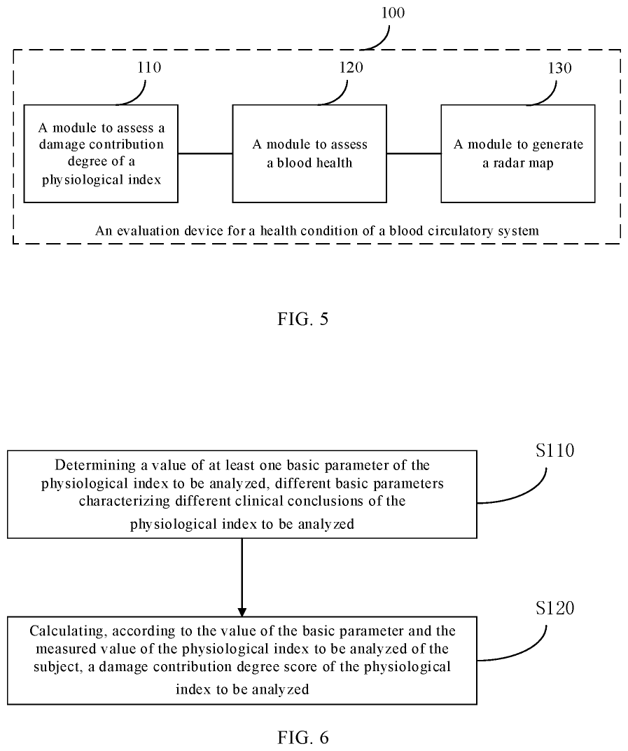

Accordingly, as shown in FIG. 5, the evaluation device 100 further includes:

a module to generate a radar map 130, configured to generate a blood health radar map according to each of the blood health state indexes and the blood health indexes.

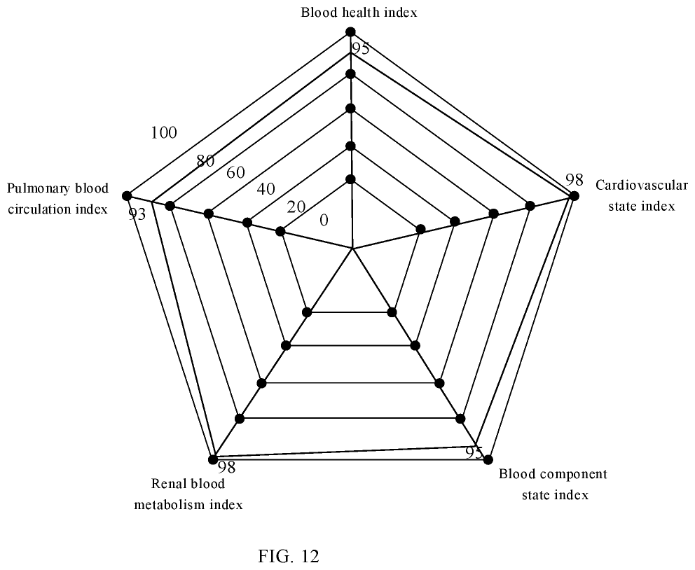

FIG. 12 shows a schematic diagram of the blood health radar map when the blood health state index includes a pulmonary blood circulation index, a cardiovascular state index, a blood component state index, and a renal blood metabolism index. The dotted line is a reference line, and the solid line is a blood health radar map of the subject.

As a second aspect of the present disclosure, there is provided an assessment method for a damage contribution degree of a physiological index, which, as shown in FIG. 6, includes the following steps S110 to S120.

At step S110, determining a value of at least one basic parameter of the physiological index to be analyzed, the different basic parameters characterizing the different clinical conclusions of the physiological index to be analyzed.

At step S120, calculating, according to the value of the basic parameter and the measured value of the physiological index to be analyzed of the subject, a damage contribution degree score of the physiological index to be analyzed of the subject, the damage contribution degree score characterizing the contribution degree of the physiological index to be analyzed to the health damage of the subject.

Each of the physiological indexes has a corresponding normal value range. When the measured value falls within the normal value range, it indicates that the physiological index is normal; and when the measured value deviates from the normal value range, it indicates that the physiological index is abnormal. When the health of the subject is damaged, the health damage degree of the subject is evaluated by measuring various physiological indexes. For each of the physiological indexes, the different physiological indexes may not have the same contribution degree to the health damage of the subject, and the different measurement results of the same physiological index may also have different levels of contribution degree to the health damage of the subject. In the present disclosure, the damage contribution degree score of a physiological index is defined to characterize the contribution degree of the physiological index to the health damage of the subject when the value of the physiological index changes; and the basic parameters for calculating the damage contribution degree score are also defined herein so that the damage contribution degree score of the physiological index to be analyzed can be calculated based on the values of the basic parameters, and thus the contribution degree of the physiological index to be analyzed to the health damage of the subject can be visually assessed through the damage contribution degree score.

The different physiological indexes have a respective corresponding normal value range, and the characteristics of different physiological indexes have different clinical significances. Therefore, the corresponding assignment needs to be performed on each basic parameter for each of the physiological indexes. In step S110, the value of each basic parameter of the physiological index to be analyzed is determined in combination with the clinical significance corresponding to the basic parameter. Further in step S120, a damage contribution degree score of the physiological index is calculated according to the value of the basic parameter and the measured value of the physiological index to be analyzed.

It should be noted that, in the assessment of the health damage contribution degree of the physiological index, the basic parameters for calculating the damage contribution degree score may be flexibly selected in combination with the clinical significance.

In the present disclosure, the specific mode of the damage contribution degree score is not particularly limited. For example, a linear assessment mode may be adopted, which means the damage contribution degree score increases or decreases linearly with a deviation magnitude of the measured value of the physiological index to be analyzed from the normal value range; or a non-linear assessment mode may be adopted, which means the damage contribution degree score increases or decreases non-linearly with the deviation magnitude of the measured value of the physiological index to be analyzed from the normal value range.

In the assessment method for the damage contribution degree of a physiological index provided in the present disclosure, the damage contribution degree score is calculated by combining the measured value of the physiological index and the basic parameter value determined according to the clinical significance, such that contribution degree of each of the physiological indexes to the health damage of the subject can be visually analyzed and assessed, and the clinical significance of each of the physiological indexes can be more clearly understood.

As described above, each of the physiological indexes has a corresponding normal value range, and when the measured value falls within the normal value range, it indicates that the physiological index of the subject is normal, so this physiological index does not contribute to the health damage of the subject. When the measured value falls outside the normal value range, it indicates that the physiological index of the subject is abnormal, so this physiological index contributes to the health damage of the subject. Therefore, it is clinically significant to divide the range during which the value of the physiological index may vary into a healthy interval and a non-healthy interval, and to assess the contribution degree of the physiological index to the health damage of the subject on this basis.

Accordingly, as an optional implementation, the basic parameter includes a top value and a bottom value;

values of the physiological index to be analyzed are distributed in a plurality of intervals, including a healthy interval and a plurality of non-healthy intervals,

the top value is an upper limit of the healthy interval; and

the bottom value is a lower limit of the healthy interval.

The inventor of the present disclosure has studied and found that the different intervals of the physiological index reflect different clinical conclusions. Taking the systolic blood pressure as an example, the interval between 90 and 120 mmHg represents a normal blood pressure, the interval between 140 and 160 mmHg represents mild hypertension, the interval between 160 and 180 mmHg represents moderate hypertension, the interval greater than 180 mmHg represents severe hypertension, and the interval less than 90 mmHg represents hypotension. It may be further refined, for example, a too low interval represents potential shock. Therefore, the present disclosure may, on the basis of dividing the range during which the value of the physiological index may vary into a healthy interval and a non-healthy interval, further use the relevant characteristic of the intervals representing different clinical conclusions as the basic parameters for calculating the damage contribution degree score.

Optionally, the basic parameter further includes an increased region and a decreased region;

the increased region represents a difference between the upper limit value and the lower limit value in a non-healthy interval of which the lower limit value is above the upper limit of the healthy interval; and

the decreased region represents a difference between the upper limit value and the lower limit value in a non-healthy interval of which the upper limit value is below the lower limit of the healthy interval.

It should be noted that the lengths of each of the intervals above the healthy interval corresponding to the different clinical conclusions maybe not the same as the lengths of each of the intervals below the healthy interval corresponding to the different clinical conclusions. Therefore, in the present disclosure, the increased region and the decreased region are assigned with values respectively, and the values of the increased region and the decreased region may be different or may be the same according to the different clinical conclusions of different physiological indexes. Therefore, the different clinical conclusions characterized by different physiological indexes in different value intervals can be more accurately reflected, and more accurate damage contribution degree scores can be obtained to precisely assess the contribution degree to health damage of the physiological indexes.

As an optional implementation, when the physiological index to be analyzed is systolic blood pressure, the healthy interval of the physiological index to be analyzed is [90, 120], the increased region is 20, and the decreased region is 10, in units of mmHg.

It will be appreciated that the larger the deviation magnitude of the physiological index from the normal value range, the greater the contribution degree to the health damage of the subject will be. In order to reflect the above clinical significance of the physiological index, as an optional implementation, as shown in FIG. 11, in step S120, when the damage contribution degree score is 0, it indicates that the physiological index to be analyzed does not contribute to the health damage of the subject; and

when damage contribution degree score is greater than 0, the damage contribution degree score is positively correlated with the contribution degree of the physiological index to be analyzed to the health damage of the subject.

The inventor of the present disclosure has studied and found that the contribution degree of the physiological index to the health damage of the subject is not linearly increased along with the increase of the deviation magnitude of the physiological index from the normal value range. In the present disclosure, the relationship between the deviation magnitude of the physiological index from the normal value range and the contribution degree to the health damage suffered by the subject is reflected by the non-linear increase of the damage contribution degree score.

Accordingly, as an optional implementation, as shown in FIG. 11, the damage contribution degree score increases non-linearly with increase of the deviation magnitude of the measured value from the normal value range of the physiological index to be analyzed.

As an optional implementation, the damage contribution degree score increases more rapidly as the deviation magnitude of the physiological index from the normal range increases.

As an optional implementation, in step S120, the damage contribution degree score is calculated by equation (4):

$$F = \{ \begin{matrix}1 - power ( 2 , \frac{Value - {R}_{C}}{α} ) , & Value > {R}_{C} \\ 1 - power ( 2 , \frac{{R}_{F} - Value}{β} ) , & Value < {R}_{F} \\ 0 , & {R}_{F} \leq Value \leq {R}_{C}\end{matrix}$$

where F is the damage contribution degree score, Value is the measured value, R_{C} is the top value, R_{F} is the bottom value, α is the increased region, and β is the decreased region.

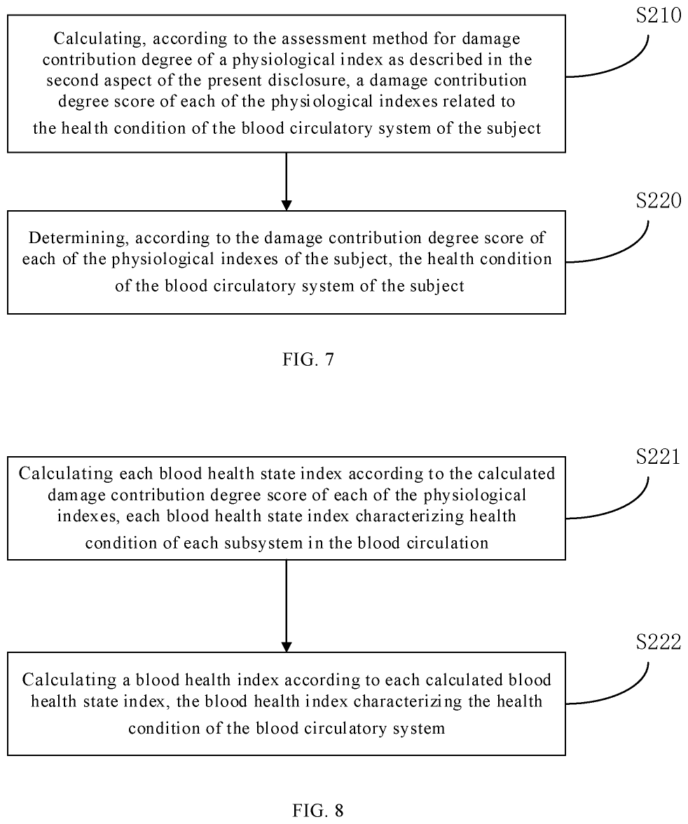

As a third aspect of the present disclosure, there is provided an assessment method for a health condition of a blood circulatory system, which, as shown in FIG. 7, includes the following steps S210 to S220:

At step S210, calculating, according to the assessment method for the damage contribution degree of a physiological index as described in the second aspect of the present disclosure, a damage contribution degree score of each of the physiological indexes related to health condition of the blood circulatory system of the subject.

At step S220, determining, according to the damage contribution degree score of each of the physiological indexes of the subject, the health condition of the blood circulatory system of the subject.

In the assessment method for the health condition of a blood circulatory system provided by the present disclosure, the health condition of the blood circulatory system of the subject is further evaluated according to the damage contribution degree score of each of the physiological indexes related to the health condition of the blood circulatory system, so that a visual and comprehensive assessment of the health condition of the blood circulatory system is achieved, and the health condition of the subject can conveniently and visually understood by themselves.

As an optional implementation, as shown in FIG. 8, the step S220 specifically includes the following steps S221 to S222:

At step S221, calculating each blood health state index according to the calculated damage contribution degree score of each of the physiological indexes, each blood health state index characterizing the health condition of each subsystem in the blood circulation.

At step S222, calculating a blood health index according to each of the calculated blood health state index, the blood health index characterizing the health condition of the blood circulatory system.

In order to comprehensively assess the health condition of the blood circulatory system, both the overall health condition of the blood circulatory system and the health condition of each subsystem in the blood circulation should be assessed. In the present disclosure, the blood health state index is defined to characterize the health condition of each subsystem in the blood circulation. The blood health state index is obtained by further calculation on the basis of the calculated damage contribution degree score of each of the physiological indexes.

The present disclosure further defines the blood health index, which characterizes the health condition of the blood circulatory system, and respective blood health indexes are calculated from the respective blood health state indexes calculated in step S222, so that the health condition of the blood circulatory system can be visually assessed through the blood health index.

According to the assessment method provided by the present disclosure, the blood health state index is calculated according to the damage contribution degree score of each of the physiological indexes related to the health condition of the blood circulatory system so that the health condition of each subsystem in the blood circulation can be visually assessed, and then the blood health index is further calculated, so that a visual and comprehensive assessment of the health condition of the blood circulatory system is achieved, and the health condition of the subject can conveniently and visually understood by themselves.

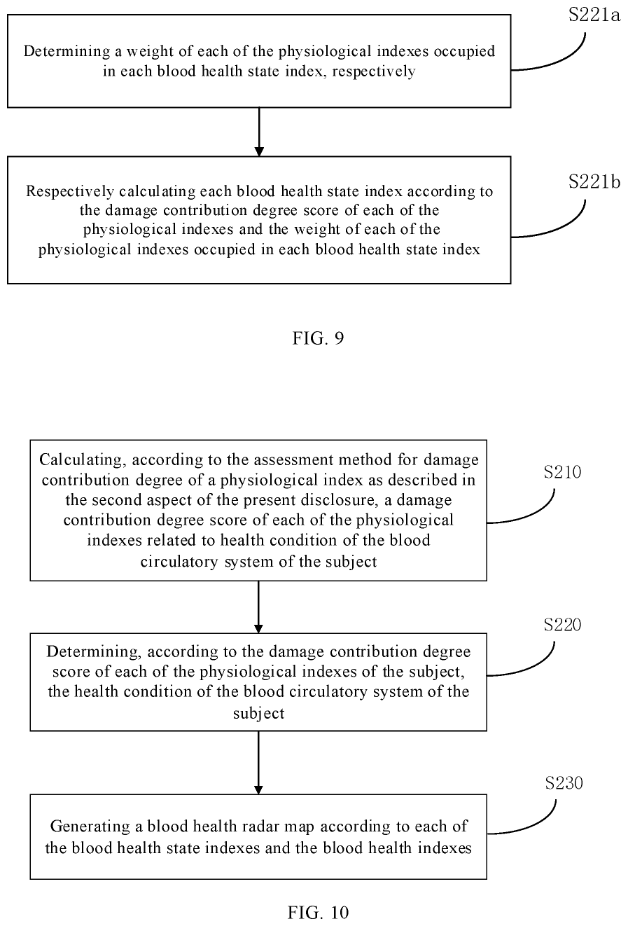

Among each of the subsystems in the blood circulation, the same physiological index may have different influences on different subsystems; and in the same subsystem, different physiological indexes may also have different influences on the subsystem. In the present disclosure, each of the physiological indexes is assigned with a weight in each subsystem in combination with the clinical significance, which reflects the weight of the damage contribution degree of each of the physiological indexes to each subsystem in the blood circulation when the subsystem suffers from health damage. Accordingly, as shown in FIG. 9, the step S221 specifically includes S221a to S221b:

At step S221a, determining a weight of each of the physiological indexes occupied in each blood health state index, respectively.

At step S221b, respectively calculating each blood health state index according to the damage contribution degree score of each of the physiological indexes and the weight of each of the physiological indexes occupied in each blood health state index.

As an optional implementation, in step S221b, the blood health state index includes a pulmonary blood circulation index, a cardiovascular state index, a blood component state index, and a renal blood metabolism index, and the blood health state index is calculated by equation (5):

Pⱼ=ΣFᵢ×ωᵢ×100 (5)

where Pⱼ is the blood health state index, Fᵢ is the damage contribution degree score of the physiological index, ωᵢ is the weight of the physiological index Fᵢ occupied in the blood health state index Pⱼ, i and j are positive integers, the different values of i correspond to different physiological indexes, and the different values of j correspond to different blood health state indexes.

After calculating the pulmonary blood circulation index, the cardiovascular state index, the blood component state index, or the renal blood metabolism index in step S221b, the blood health index is calculated by equation (6) in step S222:

BHI=[2×min(P₁, P₂, P₃, P₄)+P₁+P₂+P₃+P₄]/6 (6)

where BHI is a blood health index, P₁ is a pulmonary blood circulation index, P₂ is a cardiovascular state index, P₃ is a blood component state index, and P₄ is a renal blood metabolism index.

In the present disclosure, the health condition of the blood circulatory system is more visually presented by constructing a blood health radar map.

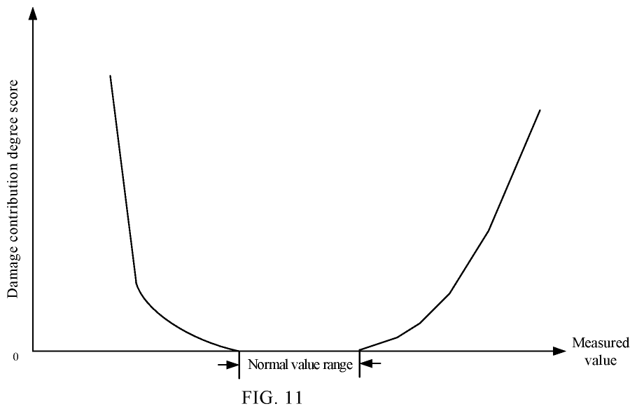

Accordingly, in addition to steps S210 to S220, the assessment method for blood health provided by the present disclosure further includes step S230 after step S220 as shown in FIG. 10:

At step S230, generating a blood health radar map according to each of the blood health state indexes and the blood health indexes.

FIG. 12 shows a schematic diagram of a blood health radar map when the blood health state index includes a pulmonary blood circulation index, a cardiovascular state index, a blood component state index, and a renal blood metabolism index. The dotted line is a reference line, and the solid line is a blood health radar map of the subject.

It will be appreciated that the above implementations are merely exemplary implementations for the purpose of illustrating the principle of the disclosure, and the present disclosure is not limited thereto. It will be apparent to those skilled in the art that various modifications and variations can be made to the invention without departing from the spirit or essence of the invention. Such modifications and variations should also be considered as falling into the protection scope of the invention.

1. An evaluation device for a health condition of a blood circulatory comprising:
a module to assess a damage contribution degree of a physiological index, configured to calculate, according to a measured value of a physiological index to be analyzed of a subject, a damage contribution degree score of the physiological index to be analyzed, the damage contribution degree score characterizing the contribution degree of the physiological index to be analyzed to a health damage of the subject; and
a module to assess a blood health, configured to determine, according to the damage contribution degree score of each of the physiological indexes related to the health condition of a blood circulatory system of the subject that is calculated and obtained by the module to assess a damage contribution degree of a physiological index, the health condition of the blood circulatory system of the subject.

2. The evaluation device according to claim 1, wherein the module to assess a damage contribution degree of a physiological index comprises:
a basic parameter assignment unit configured to determine a value of at least one basic parameter of the physiological index to be analyzed, different basic parameters characterizing different clinical conclusions of the physiological index to be analyzed; and
a damage contribution degree score calculation unit configured to calculate, according to the value of the basic parameter and the measured value of the physiological index to be analyzed of the subject, a damage contribution degree score of the physiological index to be analyzed of the subject.

3. The evaluation device according to claim 2, wherein the basic parameter comprises a top value and a bottom value;
the values of the physiological index to be analyzed are distributed in a plurality of intervals comprising a healthy interval and a plurality of non-healthy intervals,
the top value is an upper limit of the healthy interval; and
the bottom value is a lower limit of the healthy interval.

4. The evaluation device according to claim 3, wherein the basic parameter further comprises an increased region and a decreased region;
the increased region represents a difference between an upper limit value and a lower limit value in the non-healthy interval of which the lower limit value is above the upper limit of the healthy interval; and
the decreased region represents a difference between an upper limit value and a lower limit value in the non-healthy interval of which the upper limit value is below the lower limit of the healthy interval.

5. The evaluation device according to claim 2, wherein the damage contribution degree score increases non-linearly with increase of a deviation magnitude of the measured value from a normal value range of the physiological index to be analyzed.

6. The evaluation device according to claim 4, wherein the damage contribution degree score is calculated by the following equation:
$$F = \{ \begin{matrix}1 - power ( 2 , \frac{Value - {R}_{C}}{α} ) , & Value > {R}_{C} \\ 1 - power ( 2 , \frac{{R}_{F} - Value}{β} ) , & Value < {R}_{F} \\ 0 , & {R}_{F} \leq Value \leq {R}_{C}\end{matrix}$$
where F is the damage contribution degree score, Value is the measured value, R_{C} is the top value, R_{F} is the bottom value, α is the increased region, and β is the decreased region.

7. The evaluation device according to claim 1, wherein the module to assess a blood health comprises:
a blood health state index calculation unit configured to calculate each of the blood health state indexes according to the calculated damage contribution degree score of each of the physiological indexes, each of the blood health state indexes characterizing a health condition of each subsystem in the blood circulation; and
a blood health index calculation unit configured to calculate a blood health index according to each of the calculated blood health state index, the blood health index characterizing the health condition of the blood circulatory system.

8. The evaluation device according to claim 7, wherein the blood health state index calculation unit comprises:
a weight assignment subunit configured to determine a weight of each of the physiological indexes occupied in each of the blood health state indexes, respectively; and
a blood health state index calculation subunit configured to respectively calculate each of the blood health state indexes according to the damage contribution degree score of each of the physiological indexes and the weight of each of the physiological indexes occupied in each of the blood health state indexes.

9. The evaluation device according to claim 8, wherein the blood health state index comprises a pulmonary blood circulation index, a cardiovascular state index, a blood component state index, and a renal blood metabolism index, and the blood health state index is calculated by the following equation:
Pⱼ=ΣFᵢ×ωᵢ×100
where Pⱼ is the blood health state index, Fᵢ is the damage contribution degree score of the physiological index, ωᵢ is the weight of the physiological index Fᵢ occupied in the blood health state index Pᵢ, i and j are positive integers, different values of i correspond to different physiological indexes, and different values of j correspond to different blood health state indexes.

10. The evaluation device according to claim 8, wherein the blood health state index comprises a pulmonary blood circulation index, a cardiovascular state index, a blood component state index, and a renal blood metabolism index, and the blood health index is calculated by the following equation:
BHI=[2×min(P₁, P₂, P₃, P₄)+P₁+P₂+P₃+P₄]/6
where BHI is the blood health index, P₁ is the pulmonary blood circulation index, P₂ is the cardiovascular state index, P₃ is the blood component state index, and P₄ is the renal blood metabolism index.

11. An assessment method for a damage contribution degree of a physiological comprising steps of:
determining a value of at least one basic parameter of the physiological index to be analyzed, different basic parameters characterizing different clinical conclusions of the physiological index to be analyzed; and
calculating, according to the value of the basic parameter and the measured value of the physiological index to be analyzed of a subject, a damage contribution degree score of the physiological index to be analyzed of the subject, the damage contribution degree score characterizing a contribution degree of the physiological index to be analyzed to a health damage of the subject.

12. The assessment method according to claim 11, wherein the basic parameter comprises a top value and a bottom value;
the values of the physiological index to be analyzed are distributed in a plurality of intervals, comprising a healthy interval and a plurality of non-healthy intervals,
the top value is an upper limit of the healthy interval; and
the bottom value is a lower limit of the healthy interval.

13. The assessment method according to claim 12, wherein the basic parameter further comprises an increased region and a decreased region;
the increased region represents a difference between the upper limit value and the lower limit value in the non-healthy interval of which the lower limit value is above the upper limit of the healthy interval; and
the decreased region represents a difference between the upper limit value and the lower limit value in the non-healthy interval of which the upper limit value is below the lower limit of the healthy interval.

14. The assessment method according to claim 11, wherein the damage contribution degree score increases non-linearly with increase of a deviation magnitude of the measured value from a normal value range of the physiological index to be analyzed.

15. The assessment method according to claim 13, wherein the damage contribution degree score is calculated by the following equation:
$$F = \{ \begin{matrix}1 - power ( 2 , \frac{Value - {R}_{C}}{α} ) , & Value > {R}_{C} \\ 1 - power ( 2 , \frac{{R}_{F} - Value}{β} ) , & Value < {R}_{F} \\ 0 , & {R}_{F} \leq Value \leq {R}_{C}\end{matrix}$$
where F is the damage contribution degree score, Value is the measured value, R_{C} is the top value, R_{F} is the bottom value, α is the increased region, and β is the decreased region.

16. An assessment method for a health condition of a blood circulatory system, comprising steps of:
calculating, according to the assessment method for a damage contribution degree of a physiological index according to claim 11, the damage contribution degree score of each of the physiological indexes related to a health condition of the blood circulatory system of a subject; and
determining, according to the damage contribution degree score of each of the physiological indexes of the subject, the health condition of the blood circulatory system of the subject.

17. The assessment method according to claim 16, wherein the step determining, according to the damage contribution degree score of each of the physiological indexes of the subject, the health condition of the blood circulatory system of the subject comprises:
calculating each blood health state index according to the calculated damage contribution degree score of each of the physiological indexes, each blood health state index characterizing a health condition of each subsystem in a blood circulation; and
calculating a blood health index according to each of the calculated blood health state index, the blood health index characterizing the health condition of the blood circulatory system.

18. The assessment method according to claim 17, wherein the step of calculating each blood health state index according to the calculated damage contribution degree score of each of the physiological indexes comprises:
determining a weight of each of the physiological indexes occupied in each blood health state index, respectively; and
respectively calculating each of the blood health state indexes according to the damage contribution degree score of each of the physiological indexes and the weight of each of the physiological indexes occupied in each of the blood health state index.

19. The assessment method according to claim 18, wherein the blood health state index comprises a pulmonary blood circulation index, a cardiovascular state index, a blood component state index, and a renal blood metabolism index, and the blood health state index is calculated by the following equation:
Pⱼ=ΣFᵢ×ωᵢ×100
where Pⱼ is the blood health state index, Fᵢ is the damage contribution degree score of the physiological index, ωᵢ is the weight of the physiological index Fᵢ occupied in the blood health state index P, i and j are positive integers, different values of i correspond to different physiological indexes, and different values of j correspond to different blood health state indexes.

20. The assessment method according to claim 18, wherein the blood health state index comprises a pulmonary blood circulation index, a cardiovascular state index, a blood component state index, and a renal blood metabolism index, and the blood health index is calculated by the following equation:
BHI=[2×min(P₁, P₂, P₃, P₄)+P₁+P₂+P₃+P₄]/6
where BHI is the blood health index, P₁ is the pulmonary blood circulation index, P₂ is the cardiovascular state index, P₃ is the blood component state index, and P₄ is the renal blood metabolism index.
